(19) United States
(12) Patent Application Publication
(10) Pub. No.: US 2024/0055908 A1
(43) Pub. Date: Feb. 15, 2024

(54) WIRELESS POWER RECEPTION MODULE

(71) Applicant: AMOSENSE CO.,LTD, Cheonan-si, Chungcheongnam-do (KR)

(72) Inventors: Kil Jae JANG, Cheonan-si, Chungcheongnam-do (KR); Ki Chul KIM, Cheonan-si, Chungcheongnam-do (KR)

(73) Assignee: AMOSENSE CO.,LTD, Cheonan-si, Chungcheongnam-do (KR)

(21) Appl. No.: 18/260,536

(22) PCT Filed: Jan. 7, 2022
(86) PCT No.: PCT/KR2022/000289

§ 371 Date: Jul. 6, 2023

(30) Foreign Application Priority Data
Jan. 8, 2021 (KR) 10-2021-0002612
Jan. 7, 2022 (KR) 10-2022-0002592

(51) Int. Cl. H02J 50/70; H02J 50/10; H02J 50/00; A61B 5/024

(52) U.S. Cl. CPC H02J 50/70; H02J 50/10; H02J 50/005; A61B 5/024; A61B 2560/0219; A61B 2562/182

(57) ABSTRACT

A wireless power reception module including an antenna unit which includes a circuit board including a first surface and a second surfaces which are opposite to each other, a wireless power reception antenna formed as an antenna pattern on the first surface of the circuit board, a terminal pattern extending to a predetermined length from the wireless power reception antenna on the first surface, and a first coverlay attached to the first surface and a shielding unit which includes a magnetic sheet formed of a magnetic material to shield a magnetic field and having a side surface exposed to the outside and disposed on one surface of the antenna unit to be positioned at a position corresponding to the wireless power reception antenna, the first coverlay attached to the first surface, covering both the wireless power reception antenna and part of the total length of the terminal pattern.

### CROSS-REFERENCE TO RELATED APPLICATIONS

This application is the national phase entry of International Application No. PCT/KR2022/000289, filed on Jan. 7, 2022, which is based upon and claims priority to and the benefit of Korean Patent Application No. 10-2021-0002612, filed on Jan. 8, 2021, and Korean Patent Application No. 10-2022-0002592, filed on Jan. 7, 2022, the disclosures of which are incorporated herein by reference in their entireties.

### TECHNICAL FIELD

The present invention relates to a wireless power reception module.

### BACKGROUND

Wireless power transmission technology is applied to various electronic devices due to the convenience of not needing to use wired cables for charging.

As a part of the interest, the wireless power transmission technology is being applied to a wearable device which can measure heart rate information of a user through a heart rate sensor module while worn on the user's body.

That is, the wearable device in which both the heart rate sensor module and a wireless power reception module are embedded can measure a wearer's heart rate using the heart rate sensor module and easily charge a battery with wireless power received through the wireless power reception module.

In such a wearable device, the heart rate sensor module for measuring the user's heart rate and the wireless power reception module for wireless charging are manufactured separately and electrically connected to a main board of the wearable device.

This is a factor of increasing a total thickness of the wearable device.

Accordingly, attempts to reduce the total thickness of the wearable device by integrating the heart rate sensor module and the wireless power reception module into one module are being tried.

As a part of the interest, a method of arranging a heart rate sensor module at a central portion of a wireless power reception antenna and then integrating the wireless power reception module and the heart rate sensor module into one module through a surface mounting process is being considered.

Meanwhile, in a wireless power reception module, a wireless power reception antenna is formed as an antenna pattern on a circuit board so that the wireless power reception antenna has a small thickness. In this case, the wireless power reception module, along with the wireless power reception antenna, is electrically connected to other components through a terminal pattern formed on one surface of the circuit board.

However, in the wireless power reception module, when a shielding sheet is formed of a magnetic sheet including a metallic component, a side surface of the magnetic sheet is exposed to the outside, and the terminal pattern for electrical connection with the wireless power reception antenna is formed on the same surface as a surface to which the shielding sheet is attached among both surfaces of the circuit board, there are problems as follows.

That is, when the shielding sheet is attached to one surface of the wireless power reception antenna, the side surface of the magnetic sheet exposed to the outside may be in contact with the terminal pattern formed on the same surface as a surface to which the shielding sheet is attached among the both surfaces of the circuit board, and thus there is a problem that an electrical short circuit may occur. Accordingly, there is a requirement to solve this problem.

Meanwhile, when a heart rate sensor module is disposed at a central portion of a wireless power reception antenna, and the heart rate sensor module and a wireless power reception module are integrated into one module, in a central portion of a circuit board where the wireless power reception antenna is formed as a pattern, a through part having a predetermined area should be formed so that the heart rate sensor module is disposed. Accordingly, there is a problem that a partial area, which corresponds to the through part for arranging the heart rate sensor module, of a total area of the circuit board is not used. This is a factor that increases the manufacturing cost.

### SUMMARY OF THE INVENTION

The present invention is intended to address the above-described problems and directed to providing a wireless power reception module in which an electrical short circuit problem between a magnetic sheet and a terminal pattern can be prevented even when the magnetic sheet includes a metallic component.

In addition, the present invention is also directed to providing a wireless power reception module in which a wireless power reception module and a sensor module are implemented in one module to reduce a manufacturing cost.

One aspect of the present invention provides a wireless power reception module including an antenna unit which includes a circuit board including a first surface and a second surface which are opposite to each other, a wireless power reception antenna formed as an antenna pattern on the first surface of the circuit board, a terminal pattern extending to a predetermined length from the wireless power reception antenna on the first surface of the circuit board, and a first coverlay attached to the first surface of the circuit board and a shielding unit which includes a magnetic sheet formed of a magnetic material to shield a magnetic field and having a side surface exposed to an outside and is disposed on one surface of the antenna unit to be positioned at a position corresponding to the wireless power reception antenna, wherein the first coverlay is attached to the first surface to cover both the wireless power reception antenna and a part of a total length of the terminal pattern formed on the first surface.

The magnetic sheet may be a ribbon sheet formed of an amorphous alloy or a ribbon sheet formed of a nano-crystalline alloy.

The first coverlay may be formed of an insulating material.

The shielding unit may be disposed to be positioned on one surface of the first coverlay.

The shielding unit may include a magnetic sheet having a plate shape and a predetermined area and a pair of protective films attached to both surfaces of the magnetic sheet so that a side surface of the magnetic sheet is exposed to the outside.

The wireless power reception module may include an empty space portion having a predetermined area and formed to pass through the wireless power reception module to be positioned at a central portion of the wireless power reception antenna, wherein the empty space portion may include a first empty space portion having a predetermined area and formed to pass through a central portion of the antenna unit and a second empty space portion having a predetermined area and formed to pass through a central portion of the shielding unit.

The circuit board may include a body, in which the first empty space portion is formed in a central portion of the body and the wireless power reception antenna is formed as a pattern on at least one surface of the body, and a protruding part protruding to a predetermined length from an inner edge of the body toward the first empty space portion, wherein at least a portion including one end portion in the terminal pattern may be formed on the circuit board so as to be positioned on the protruding part.

The wireless power reception module may further include a sensor module disposed in the empty space portion, wherein the sensor module may include a substrate member and a terminal pad formed on the substrate member, and the wireless power reception module and the sensor module may be integrated by the terminal pattern and the terminal pad electrically connected to each other through a solder member.

The sensor module may be a heart rate sensor module.

Another aspect of the present invention provides a wireless power reception module including a wireless power reception antenna provided with a flat coil in which an empty space portion having a predetermined area is formed in a central portion thereof and a shielding unit including a magnetic member formed of a magnetic material to shield a magnetic field, an accommodation groove formed to be recessed in one surface of the magnetic member to accommodate the wireless power reception antenna, a through part formed to pass through the magnetic member at a position corresponding to the empty space portion, and an insulating protection layer with a predetermined thickness formed on the surface of the magnetic member, wherein the magnetic member includes a base which has a closed-loop shape and in which the through part is formed in a central portion of the base, an inner protruding part protruding to a predetermined thickness from one surface of the base to surround the through part, and an outer protruding part protruding to a predetermined thickness from the one surface of the base to be spaced apart from the inner protruding part so as to form the accommodation groove in the one surface of the base.

The magnetic member may be formed of a ferrite material.

Thicknesses of the inner protruding part and the outer protruding part that protrude from the one surface of the base may be the same.

The inner protruding part may be formed to protrude from the one surface of the base to have a greater thickness than the outer protruding part.

The base, the inner protruding part, and the outer protruding part may be integrally formed.

The wireless power reception module may further include a sensor module disposed in the empty space portion, wherein both end portions of the flat coil may be integrated with a circuit board constituting the sensor module through a soldering process.

According to the present invention, even when a magnetic sheet includes a metallic component, since an electrical short circuit problem between the magnetic sheet and a terminal pattern can be prevented, the reliability of a product can be improved.

In addition, according to the present invention, even when a sensor module is disposed at a central portion of a wireless power reception antenna so that the sensor module and the wireless power reception antenna are implemented in one module, a use amount of a circuit board can be minimized, or there is no discarded portion fundamentally, and thus a manufacturing cost can be reduced.

In addition, according to the present invention, even when an empty space portion for arranging a sensor module at a central portion of a shielding unit is formed, an amount of a magnetic field leaking through the empty space portion can be minimized to increase an amount of a magnetic field concentrated on the shielding unit. Accordingly, the performance of a wireless power reception antenna can be improved.

### BRIEF DESCRIPTION OF THE DRAWINGS

FIG. 1 is a view illustrating a wireless power reception module according to one embodiment of the present invention.

FIG. 2 is a cross-sectional view illustrating the wireless power reception module along line A-A in FIG. 1 and illustrates a state in which a shielding unit attached to one surface of the antenna unit.

FIG. 3 is a view illustrating a state in which a sensor module is coupled to the wireless power reception module in FIG. 2.

FIG. 4 is a view illustrating a state in which the sensor module is coupled to the wireless power reception module in FIG. 1.

FIG. 5 is a view illustrating a wireless power reception module according to another embodiment of the present invention.

FIG. 6 is a cross-sectional view illustrating the wireless power reception module along line B-B in FIG. 5 and illustrates a state in which a shielding unit is attached to one surface of an antenna unit.

FIG. 7 is a view illustrating a state in which a sensor module is coupled to the wireless power reception module in FIG. 6.

FIG. 8 is a view illustrating a wireless power reception module according to still another embodiment of the present invention.

FIG. 9 is a cross-sectional view illustrating the wireless power reception module along line C-C of FIG. 8 and illustrates a state in which a shielding unit is attached to one surface of an antenna unit.

FIG. 10 is a view illustrating a state in which a sensor module is coupled to the wireless power reception module in FIG. 9.

FIG. 11 is a view illustrating a wireless power reception module according to yet another embodiment of the present invention.

FIG. 12 is a cross-sectional view illustrating the wireless power reception module along line D-D of FIG. 11.

FIG. 13 is a view illustrating a wireless power reception module according to yet another embodiment of the present invention.

FIG. 14 is a cross-sectional view illustrating the wireless power reception module along line E-E of FIG. 13.

FIG. 15 is a view illustrating a wireless power reception module according to yet another embodiment of the present invention.

FIG. 16 is a bottom view illustrating the wireless power reception module in FIG. 15.

FIG. 17 is a cross-sectional view illustrating the wireless power reception module along line F-F of FIG. 15.

FIG. 18 is a view illustrating a cross section of another magnetic member applicable to the wireless power reception module in FIG. 15.

### DETAILED DESCRIPTION

Hereinafter, various embodiments of the present invention will be described in detail with reference to the accompanying drawings in order for those skilled in the art to easily perform the present invention. The present invention may be implemented in several different forms and is not limited to the embodiments described herein. Parts irrelevant to description are omitted in the drawings in order to clearly describe the present invention, and the same part or similar parts are denoted by the same reference numeral throughout this specification.

A wireless power reception module 100, 200, or 300 according to one embodiment of the present invention may receive wireless power transmitted from the outside.

As an example, the wireless power reception module 100, 200, or 300 according to one embodiment of the present invention may be applied to a wearable device such as a smart watch.

That is, the wireless power reception module 100, 200, or 300 according to one embodiment of the present invention may be embedded in the wearable device and may receive the wireless power transmitted from the outside to charge a battery of the wearable device.

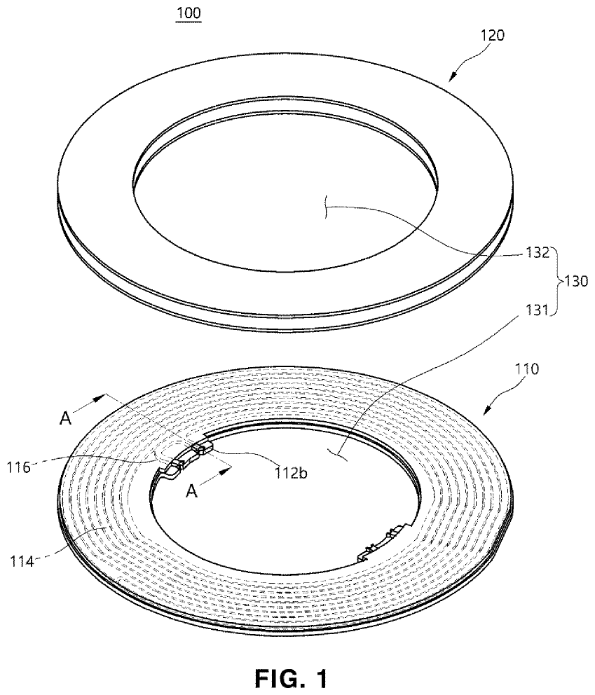

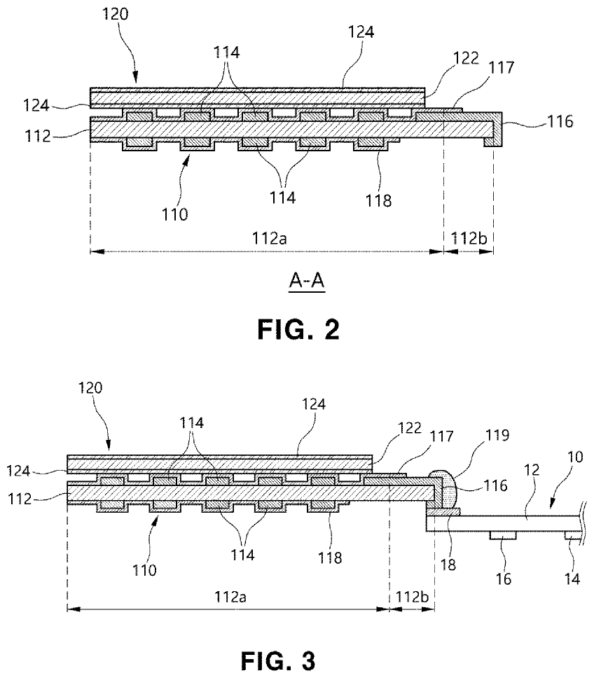

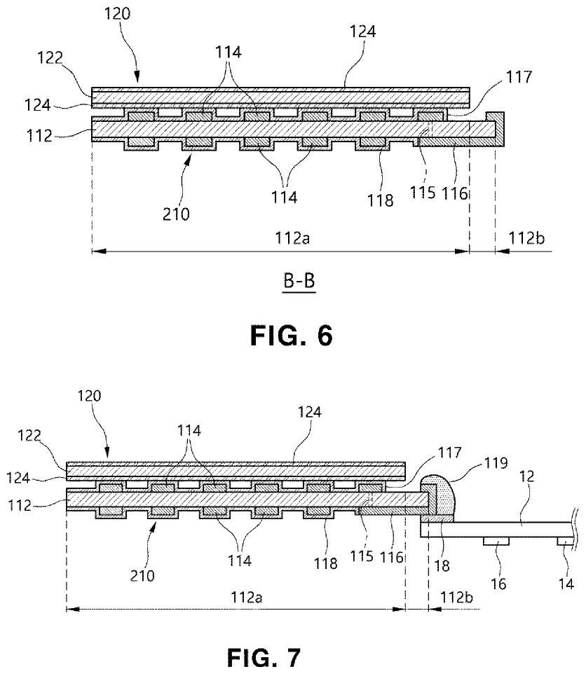

In this case, the wireless power reception module 100, 200, or 300 according to one embodiment of the present invention may be electrically connected to a sensor module 10, which differs from the wireless power reception module 100, 200, or 300, through a terminal pattern 116 as illustrated in FIGS. 1 to 10. Accordingly, the wireless power reception module 100, 200, or 300 according to one embodiment of the present invention may be integrated with the sensor module 10.

That is, in the wireless power reception module 100, 200, or 300 according to one embodiment of the present invention, the terminal pattern 116 may be electrically connected to a terminal pad 18 of a substrate member 12 constituting the sensor module 10 through a solder member 119.

Accordingly, the wireless power reception module 100, 200, or 300 according to one embodiment of the present invention may be integrated with the sensor module 10 through a surface mounting technology (SMT) method.

As a non-restrictive example, the wireless power reception module 100, 200, or 300 according to one embodiment of the present invention may be integrated with the sensor module 10 embedded in the wearable device.

In this case, the sensor module 10 may be a heart rate sensor module, and the wireless power reception module 100, 200, or 300 may be electrically connected to the terminal pad 18 of the substrate member 12 constituting the heart rate sensor module through the terminal pattern 116 and the solder member 119, and the wireless power reception module 100, 200, or 300 may be integrated with the heart rate sensor module through the SMT method.

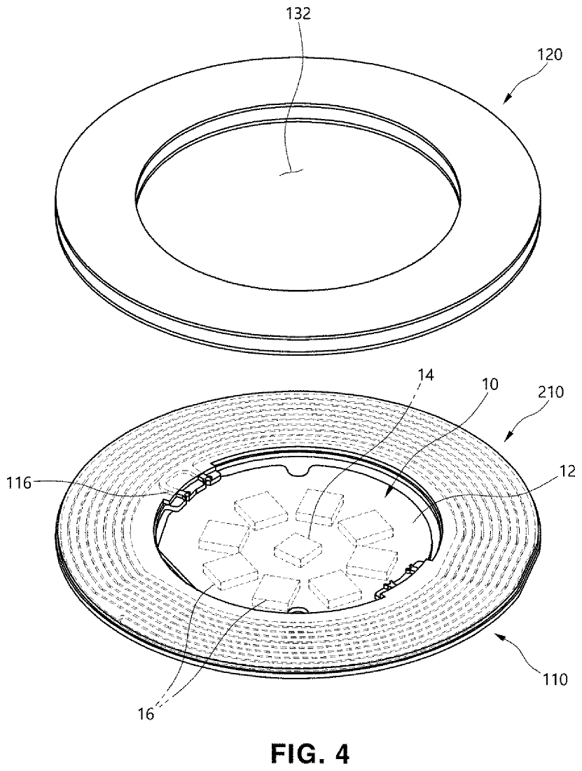

Herein, as illustrated in FIG. 4, the heart rate sensor module may be a photoplethysmography heart rate sensor module including the substrate member 12 having a plate shape and a predetermined area and a light-emitting diode (LED) 14 and a photodiode 16 which are mounted on one surface of the substrate member 12.

In addition, in the heart rate sensor module, the photodiode 16 may be provided as a plurality of photodiodes 16, and the plurality of photodiodes 16 may be mounted on one surface of the substrate member 12 to surround the LED 14.

In addition, the substrate member 12 may include the terminal pad 18 formed on one surface of the substrate member 12 for electrical connection with another module.

However, the sensor module 10 is not limited to the above-described heart rate sensor module and may be any known sensor module according to a target function.

In this case, the wireless power reception module 100, 200, or 300 may include an empty space portion 130 having a predetermined area and formed to pass through the wireless power reception module 100, 200, or 300 so that the sensor module 10 is disposed in a central portion of the wireless power reception module 100, 200, or 300.

Accordingly, as illustrated in FIG. 4, the sensor module 10 may be disposed to be positioned at a side of the empty space portion 130 formed in the central portion of the wireless power reception module 100, and the wireless power reception module 100 may be disposed to surround a perimeter of the sensor module 10.

In addition, the terminal pattern 116 formed on a circuit board 112 constituting the wireless power reception module 100 may be connected to the terminal pad 18 of the substrate member 12 constituting the sensor module 10 through the solder member 119.

Accordingly, the wireless power reception module 100 according to one embodiment of the present invention may be embedded in the wearable device while being integrated into one module with the sensor module 10, and electrically connected to a main board of the wearable device.

Accordingly, even when both the wireless power reception module 100, 200, or 300 for wireless charging and the sensor module 10 having a predetermined function, for example, a function for checking a heart rate of a user are embedded in the wearable device, the wearable device can be implemented as a thin type wearable device.

In FIG. 4, although it is illustrated that the wireless power reception module 100 illustrated in FIGS. 1 to 3 is integrated with the sensor module 10, but the present invention is not limited thereto.

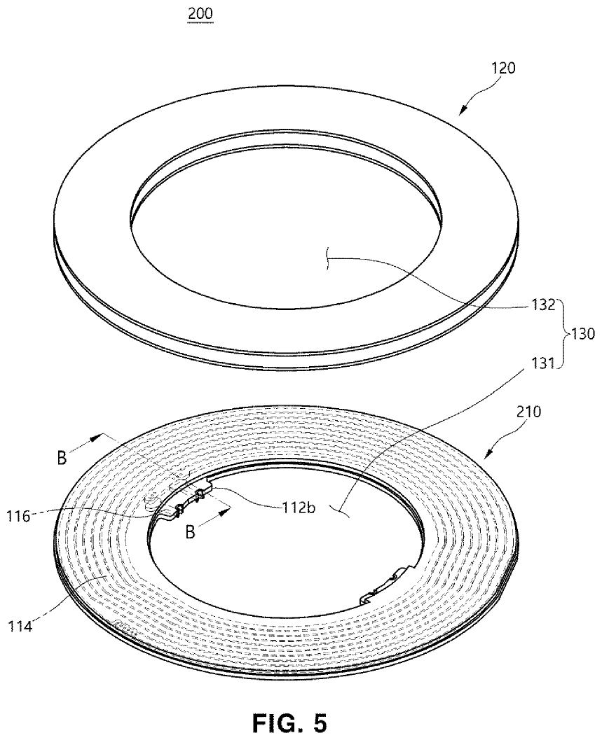

That is, like the wireless power reception module 100 illustrated in FIG. 4, the wireless power reception module 200 or 300 illustrated in FIGS. 5 to 10 may be integrated with the sensor module 10.

Meanwhile, the wireless power reception module 100, 200, or 300 according to one embodiment of the present invention may include an antenna unit 110 or 210 and a shielding unit 120 or 220, as illustrated in FIGS. 1 to 10.

The antenna unit 110 or 210 may include a wireless power reception antenna 114 for receiving the wireless power transmitted from the outside, and the shielding unit 120 or 220 may include a magnetic sheet 122 formed of a magnetic material to shield a magnetic field.

In this case, the shielding unit 120 or 220 may be disposed on one surface of the antenna unit 110 or 210 to be positioned at a position corresponding to the wireless power reception antenna 114.

In addition, as described above, the wireless power reception module 100, 200, or 300 according to one embodiment of the present invention may include the empty space portion 130 having the predetermined area and formed to pass thorough the wireless power reception module 100, 200, or 300 so that the sensor module 10 is disposed at the central portion.

As an example, the empty space portion 130 may include a first empty space portion 131 having a predetermined area and formed to pass through a central portion of the antenna unit 110 or 210 and a second empty space portion 132 having a predetermined area and formed to pass through a central portion of the shielding unit 120 or 220.

Accordingly, each of the antenna unit 110 or 210 and the shielding unit 120 or 220 may be formed in a closed-loop shape that is a ring shape. In addition, when the shielding unit 120 or 220 is disposed on one surface of the antenna unit 110 or 210, the second empty space portion 132 may be disposed to be positioned above the first empty space portion 131.

In this case, the antenna unit 110 or 210 may include the circuit board 112 including a first surface and a second surface which are opposite to each other and the wireless power reception antenna 114 formed as an antenna pattern having a loop shape on at least one surface of the circuit board 112.

In addition, the antenna unit 110 or 210 may include the terminal pattern 116 formed on the circuit board 112 to extend to a predetermined length from the wireless power reception antenna 114 and a pair of coverlays 117 and 118 attached to the first surface and the second surface of the circuit board 112, respectively, to cover the wireless power reception antenna 114.

Herein, each of the pair of coverlays 117 and 118 may be formed of an insulating material. Based on FIGS. 1 to 10, the first surface may be an upper surface of the circuit board 112, and the second surface may be a lower surface of the circuit board 112.

In addition, the pair of coverlays 117 and 118 may include a first coverlay 117 attached to the circuit board 112 to cover the first surface and a second coverlay 118 attached to the circuit board 112 to cover the second surface.

Accordingly, the wireless power reception antenna 114 may be electrically connected to another component disposed in the first empty space portion 131, such as the sensor module 10, through the terminal pattern 116, and the wireless power reception antenna 114 may be electrically insulated by the pair of coverlays 117 and 118.

In addition, the shielding unit 120 or 220 may include the magnetic sheet 122 having the plate shape and formed of the magnetic material to shield the magnetic field and at least one protective film 124 or 224a and 224b attached to at least one surface of the magnetic sheet 122 to protect the magnetic sheet 122 from an external force.

In this case, the first coverlay 117 may be disposed to be positioned between a lower surface of the shielding unit 120 or 220 and the first surface of the circuit board 112.

Herein, a Mn—Zn ferrite sheet, a Ni—Zn ferrite sheet, a ribbon sheet formed of an amorphous or nano-crystalline alloy, a polymer sheet, a permalloy, or the like may be used as the magnetic sheet 122.

In addition, the magnetic sheet 122 may be a multilayer sheet in which a plurality of sheets are stacked with adhesive layers interposed therebetween and may be formed to be separated into a plurality of pieces to increase an overall resistance so as to suppress the generation of an eddy current or improve flexibility.

In addition, the magnetic sheet 122 may have a saturation magnetic flux density is 0.4 Tesla or greater and have a thickness of 0.03 to 1.0 mm.

Herein, the material, characteristics, and thickness of the magnetic sheet 122 are not limited thereto, and any known magnetic material used as a shielding material to shield a magnetic field may be used as the magnetic sheet 122, and the material, characteristics, and thickness of the magnetic sheet 122 may be appropriately changed according to design conditions.

As a specific example, as illustrated in FIGS. 1 to 10, the wireless power reception antenna 114 may have an antenna pattern having a loop shape and formed on each of the first surface and the second surface of the circuit board 112, and the antenna pattern having the loop shape and formed on each of the first surface and the second surface of the circuit board 112 may be covered by the first coverlay 117 and the second coverlay 118 attached to the first surface and the second surface of the circuit board 112, respectively.

Herein, the antenna pattern formed on the first surface of the circuit board 112 and the antenna pattern formed on the second surface of the circuit board 112 may be electrically connected to each other through at least one via hole (not shown).

Accordingly, the wireless power reception antenna 114 may be prevented from being exposed to the outside by the pair of coverlays 117 and 118, and the wireless power reception antenna 114 may be prevented from being electrically short-circuited to other components by the insulating characteristic of the pair of coverlays 117 and 118.

In this case, one end portion of the terminal pattern 116 formed on the circuit board 112 for electrically connecting the wireless power reception antenna 114 and the other component may be formed to be electrically connected to the wireless power reception antenna 114 on the first surface or the second surface of the circuit board 112.

In this case, the terminal pattern 116 may be formed on the circuit board 112 so that at least a partial length of the total length of the terminal pattern 116 is exposed to the outside.

Accordingly, the terminal pattern 116 may be electrically connected to the terminal pad 18 of the substrate member 12 constituting the other component through a portion exposed to the outside and the solder member 119 as illustrated in FIGS. 3, 7, and 10.

Herein, the circuit board 112 may include a body 112a, in which the first empty space portion 131 is formed in a central portion of the body 112a and the wireless power reception antenna 114 is formed as a pattern on at least one surface of the body 112a, and a protruding part 112b protruding to a predetermined length from an inner edge of the body 112a toward the first empty space portion 131.

In addition, At least a portion including one end portion in the terminal pattern 116 may be disposed to be positioned on the protruding part 112b, and the other end portion of the terminal pattern 116 may be electrically connected to the wireless power reception antenna 114.

Accordingly, even when the sensor module 10 is disposed at the empty space portion 130, an area in which the wireless power reception module 100, 200, or 300 according to one embodiment of the present invention overlap the sensor module 10 can be minimized, and the wireless power reception module 100, 200, or 300 can be easily electrically connected to the sensor module 10 through the terminal pattern 116 disposed on the protruding part 112b.

In this case, even when the magnetic sheet 122 formed of the magnetic material including the metallic component is disposed on one surface of the antenna unit 110 or 210, the wireless power reception module 100, 200, or 300 according to one embodiment of the present invention may be formed to prevent an electrical short circuit between the magnetic sheet 122 and the terminal pattern 116.

As an example, as illustrated in FIGS. 1 to 3, the lower surface of the shielding unit 120 may be disposed on one surface of the antenna unit 110 to face one surface of the first coverlay 117, a side surface of the magnetic sheet 122 including the metallic component in the shielding unit 120 may be exposed to the outside, the terminal pattern 116 may be formed on the first surface of the circuit board 112 to extend from an end portion of the wireless power reception antenna 114 toward the protruding part 112b to the predetermined length.

In this case, the first coverlay 117 may be attached to the first surface of the circuit board 112 to cover both the wireless power reception antenna 114 formed as a pattern on the first surface of the circuit board 112 and a length of a part, which includes a portion extending from the end portion of the wireless power reception antenna 114, of the total length of the terminal pattern 116.

That is, the terminal pattern 116 may include a portion covered by the first coverlay 117 and a portion exposed to the outside without being covered by the first coverlay 117.

Accordingly, in a process of attaching the shielding unit 120 to one surface of the first coverlay 117, even when the shielding unit 120 is pressed to increase an adhesion and bonding force between the antenna unit 110 and the shielding unit 120, the side surface of the magnetic sheet 122 exposed to the outside may not be in direct contact with the terminal pattern 116 due to a portion of the first coverlay 117 which covers a partial length of the terminal pattern 116.

Accordingly, even when burrs or particles are present on the side surface of the magnetic sheet 122 exposed to the outside, the burr or particle may not be in direct contact with the terminal pattern 116 due to the portion of the first coverlay 117 which covers a part of the terminal pattern 116.

As a result, even when the side surface of the magnetic sheet 122 including the metallic component is exposed to the outside, in the wireless power reception module 100 according to one embodiment of the present invention, an electrical short circuit between the magnetic sheet 122 and the terminal pattern 116 due to the burrs or particles which may be present on the side surface of the magnetic sheet 122 can be prevented.

That is, when the side surface of the magnetic sheet 122 formed of the magnetic material including the metallic component in the shielding unit 120 is exposed to the outside, and the terminal pattern 116 for electrical connection with the other component is formed on the circuit board 112 to be positioned on one surface of the antenna unit 110 to which the shielding unit 120 is attached, the first coverlay 117 disposed between one surface of the shielding unit 120 and one surface of the circuit board 112 which face each other may be formed to cover the partial length of the total length of the terminal pattern 116.

Accordingly, the terminal pattern 116 can be smoothly electrically connected to the other component through a portion not covered by the first coverlay 117, and the terminal pattern 116 can be prevented from being in direct contact with the side surface of the magnetic sheet 122 exposed to the outside by a portion covered by the first coverlay 117.

In addition, even if a part of the magnetic sheet 122 is disposed on one surface of the antenna unit 110 so that it is positioned above the terminal pattern 116, the part of the magnetic sheet 122 positioned above the terminal pattern 116 may be positioned above the portion covered by the first coverlay 117.

Accordingly, the side surface of the magnetic sheet 122 exposed to the outside may be prevented from being in direct contact with the terminal pattern 116 by the portion covered by the first coverlay 117.

As another example, as illustrated in FIGS. 5 to 7, the lower surface of the shielding unit 120 may be disposed on one surface of the antenna unit 210 to face one surface of the first coverlay 117, the side surface of the magnetic sheet 122 including the metallic component in the shielding unit 120 may be exposed to the outside, and the terminal pattern 116 may be formed on the second surface of the circuit board 112 which is opposite to the first surface of the circuit board 112 to which the first coverlay 117 is attached.

That is, the terminal pattern 116 may be formed on the second surface of the circuit board 112 to extend from the body 112a toward the protruding part 112b to the predetermined length, and a portion of the terminal pattern 116 formed on the body 112a may be electrically connected to the wireless power reception antenna 114 formed on the first surface of the circuit board 112 through the via hole 115.

In this case, the first coverlay 117 may be attached to the first surface of the circuit board 112 to cover the entire wireless power reception antenna 114 which is formed as a pattern on the first surface of the circuit board 112, and the second coverlay 118 may be attached to the second surface of the circuit board 112 so as not to cover at least a partial length of the total length of the terminal pattern 116.

Accordingly, in a process of attaching the shielding unit 120 to one surface of the first coverlay 117, even when the shielding unit 120 is pressed to increase an adhesion and bonding force between the antenna unit 210 and the shielding unit 120, the side surface of the magnetic sheet 122 exposed to the outside may be fundamentally prevented from being in direct contact with the terminal pattern 116 formed on the second surface of the circuit board 112.

That is, in the present embodiment, even when the side surface of the magnetic sheet 122 formed of the magnetic material including the metallic component is exposed to the outside, the terminal pattern 116 for electrical connection with the other component may be formed on the second surface of the circuit board 112 so that the terminal pattern 116 is positioned on a surface opposite to one surface of the antenna unit 210 to which the shielding unit 120 is attached.

Accordingly, in the wireless power reception module 200 according to one embodiment of the present invention, the side surface of the magnetic sheet 122 exposed to the outside may be fundamentally prevented from being in direct contact with the terminal pattern 116 formed on the second surface of the circuit board 112.

As a result, even when the side surface of the magnetic sheet 122 including the metallic component is exposed to the outside, in the wireless power reception module 200 according to one embodiment of the present invention, an electrical short circuit between the magnetic sheet 122 and the terminal pattern 116 due to the burrs or particles which may be present on the side surface of the magnetic sheet 122 can be prevented.

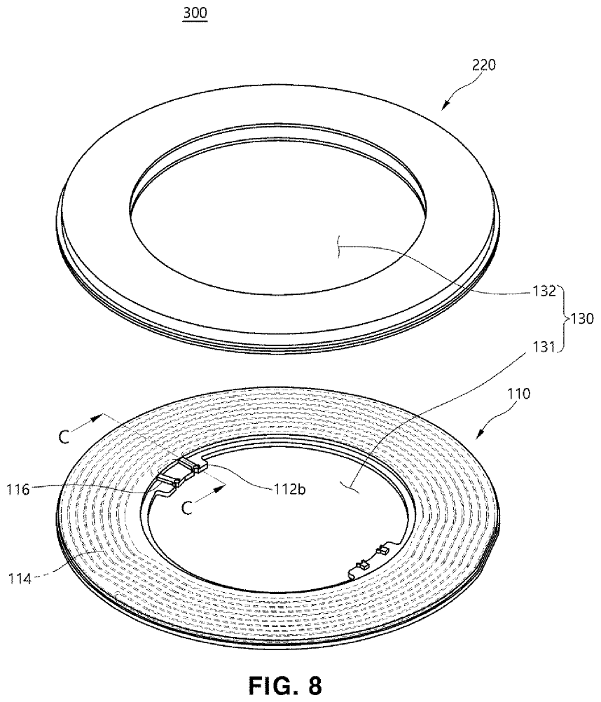

As another example, as illustrated in FIGS. 8 to 10, the lower surface of the shielding unit 220 may be disposed on one surface of the antenna unit 110 to face one surface of the first coverlay 117, the terminal pattern 116 may be formed on the first surface of the circuit board 112 to extend from the end portion of the wireless power reception antenna 114 toward the protruding part 112b to the predetermined length, and the magnetic sheet 122 may be formed so that all surfaces are not exposed to the outside.

That is, the shielding unit 220 may include the protective films 224a and 224b which are attached to the magnetic sheet 122 to cover all surfaces of the magnetic sheet 122.

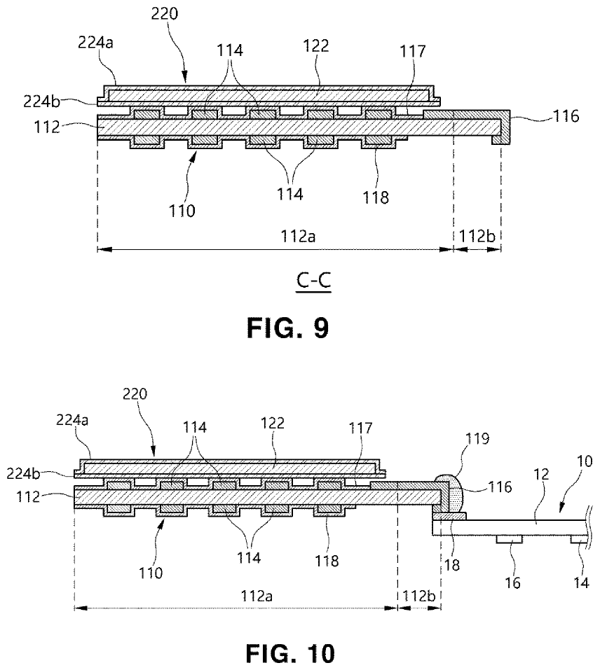

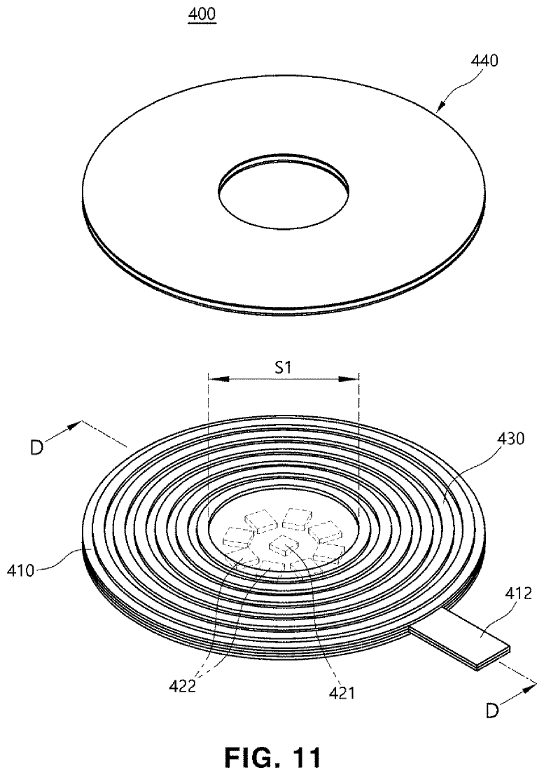

In this case, the protective films 224a and 224b may include a first protective film 224a which covers an upper surface and the side surface of the magnetic sheet 122 and a second protective film 224b which covers a lower surface of the magnetic sheet 122 as illustrated in FIGS. 9 and 10.

In addition, the first coverlay 117 may be attached to the first surface of the circuit board 112 to cover the wireless power reception antenna 114 formed as a pattern on the first surface of the circuit board 112, and the second coverlay 118 may be attached to the second surface of the circuit board 112 to cover the wireless power reception antenna 114 formed as a pattern on the second surface of the circuit board 112.

Accordingly, even when the magnetic sheet 122 is formed of the magnetic material including the metallic component, in the wireless power reception module 300 according to one embodiment of the present invention, the metallic component included in the magnetic sheet 122 can be fundamentally prevented from being exposed to the outside or separated from the magnetic sheet 122 by the protective films 224a and 224b which surround all the surfaces of the magnetic sheet 122.

As a result, even when the magnetic sheet 122 is formed of the magnetic material including the metallic component, and the terminal pattern 116 is formed on one surface of the circuit board 112 to be exposed to the outside, the magnetic sheet 122 can be prevented from being in direct contact with the terminal pattern 116 by the protective films 224a and 224b which cover all the surfaces.

Accordingly, in a process of attaching the shielding unit 220 to one surface of the first coverlay 117, even when the shielding unit 220 is pressed to increase an adhesion and bonding force between the antenna unit 110 and the shielding unit 220, the magnetic sheet 122 may be fundamentally prevented from being in direct contact with the terminal pattern 116 by the protective films 224a and 224b.

That is, even when the magnetic sheet 122 is formed of the magnetic material including the metallic component, in the wireless power reception module 300 according to one embodiment of the present invention, an electrical short circuit between the magnetic sheet 122 and the terminal pattern 116 due to the metallic component of the magnetic sheet 122 can be prevented.

In the drawings, even though it is illustrated that the first protective film 224a is attached to the magnetic sheet 122 to cover the upper surface and the side surface of the magnetic sheet 122 and the second protective film 224b is attached to the magnetic sheet 122 to cover the lower surface of the magnetic sheet 122, the present invention is not to be limited thereto, and roles of the first protective film 224a and the second protective film 224b configured to cover the surfaces of the magnetic sheet 122 may be interchanged.

As an example, the second protective film 224b may be attached to the magnetic sheet 122 to cover the lower surface and the side surface of the magnetic sheet 122, and the first protective film 224a may be attached to the magnetic sheet 122 to cover the upper surface of the magnetic sheet 122.

In addition, the first protective film 224a may be attached to the magnetic sheet 122 to cover the upper surface and a part of the side surface of the magnetic sheet 122, and the second protective film 224b may be attached to the magnetic sheet 122 to cover the lower surface and the remaining side surface of the magnetic sheet 122.

That is, in the wireless power reception module 300 according to the present embodiment, when at least one protective film may cover all the surfaces of the magnetic sheet 122, the protective film may be attached to the magnetic sheet 122 in various manners.

In addition, in the wireless power reception module 300 according to the present embodiment, it is illustrated that the terminal pattern 116 is formed on the first surface of the circuit board 112 which is coplanar with one surface of the antenna unit 110 to which the shielding unit 220 is attached, but the present invention is not limited thereto, and the terminal pattern 116 may be formed on the second surface of the circuit board 112 which is opposite to one surface of the antenna unit 110 to which the shielding unit 220 is attached as illustrated in FIGS. 5 to 7.

Meanwhile, a wireless power reception module 400, 500, or 600 according to one embodiment of the present invention may further include a sensor module 420 or 510 which serves a predetermined function.

In this case, the wireless power reception module 400, 500, or 600 and the sensor module 420 or 510 may be integrated with each other.

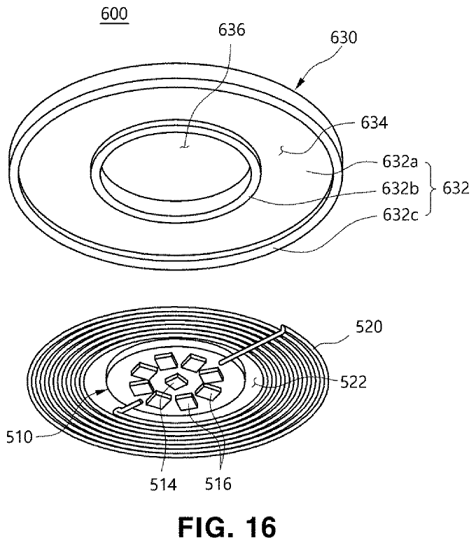

That is, as illustrated in FIGS. 11 to 17, the wireless power reception module 400, 500, or 600 according to one embodiment of the present invention may be implemented in an integrated form of a wireless power reception antenna 430 or 520 for receiving wireless power transmitted from the outside and the sensor module 420 or 510 which serves the predetermined function.

As a non-restrictive example, the wireless power reception module 400, 500, or 600 according to one embodiment of the present invention may be embedded in a wearable device in a state in which the wireless power reception antenna 430 or 520 and the sensor module 420 or 510 are integrated.

In this case, the sensor module 420 or 510 may be a heart rate sensor module, and the heart rate sensor module may be a photoplethysmography heart rate sensor module including an LED 421 or 514 and a photodiode 422 or 516.

In addition, in the heart rate sensor module, the photodiode 422 or 516 may be provided as a plurality of photodiodes 422 or 516, and the plurality of photodiodes may be disposed to surround the LED 421 or 514.

However, the sensor module 420 or 510 is not limited to the above-described heart rate sensor module and may be any known sensor module according to a target function.

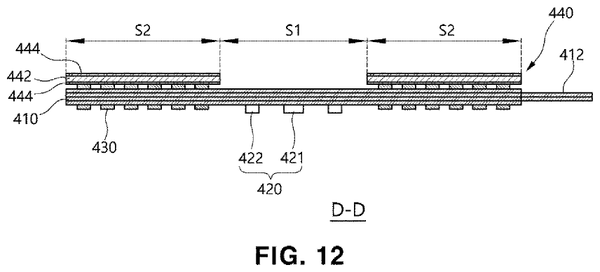

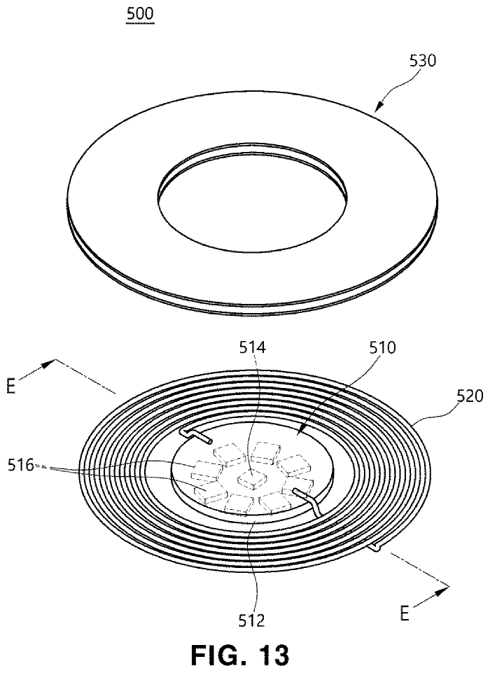

As a specific example, as illustrated in FIGS. 11 and 12, in the wireless power reception module 400 according to one embodiment of the present invention, both the wireless power reception antenna 430 and the sensor module 420 may be formed on one circuit board 410.

That is, the circuit board 410 may be formed in a plate shape having a predetermined area and may be divided into a first region S1 having a predetermined area and a second region S2 formed to surround the first region S1.

In this case, the wireless power reception antenna 430 for receiving the wireless power transmitted from the outside may be an antenna pattern formed on at least one surface of the circuit board 410 to be positioned in the second region S2, and the sensor module 420 may include the LED 421 and the photodiode 422 mounted on the circuit board 410 to be positioned in the first region S1.

In addition, a shielding unit 440 may be disposed on one surface of the circuit board 410 to be positioned at a position corresponding to the wireless power reception antenna 430.

In this case, the shielding unit 440 may include a magnetic sheet 442 having a plate shape for shielding a magnetic field and a protective film 444 attached to at least one surface of the magnetic sheet 442.

In this case, the magnetic sheet 122 and protective film 124 or 224a and 224b may be equally applied to the magnetic sheet 442 and the protective film 444.

Accordingly, in the wireless power reception module 400 according to the present embodiment, the wireless power reception antenna 430 formed in the second region S2 may be disposed in the one circuit board 410 to surround the sensor module 420 mounted on the first region S1.

Accordingly, in the wireless power reception module 400 according to the present embodiment, both the wireless power reception antenna 430 and the sensor module 420 may be formed on one circuit board 410.

That is, when the wireless power reception module 400 according to the present embodiment and the wireless power reception module 100, 200, or 300 illustrated in FIGS. 1 to 10 are compared, the total area of one circuit board 410 may be divided into a partial area corresponding to the second region S2 in which the wireless power reception antenna 430 is formed and the remaining area corresponding to the first region S1 in which the sensor module 420 is mounted.

On the other hand, the first empty space portion 131 having a predetermined area and formed to pass through the central portion is formed in the circuit board 112 on which the wireless power reception antenna 114 is formed in the wireless power reception module 100, 200, or 300 illustrated in FIGS. 1 to 10.

Accordingly, although the sensor module 10 including the LED 14, the photodiode 16, and the substrate member 12 is disposed at the first empty space portion 131 formed in the central portion of the wireless power reception antenna 114, a portion cut from the circuit board 112 to form the first empty space portion 131 may be discarded.

However, in the wireless power reception module 400 according to the present embodiment, since the total area of the circuit board 410 is divided into two regions, a portion of the circuit board 410 corresponding to the first region S1 may be used as a circuit board for constituting the sensor module 420 even without using a separate circuit board (corresponding to a reference numeral “12” in FIG. 4) for constituting the sensor module 420.

Accordingly, in the present embodiment, when compared to the above-described embodiment, a central portion (an area corresponding to the first region) which is cut out of the circuit board 112 on which the wireless power reception antenna 114 is formed and discarded in order to arrange the sensor module 10 at a central portion of the wireless power reception antenna 114 may be used instead of being discarded, and thus a cost can be reduced.

In addition, since the wireless power reception antenna 430 may be formed on one circuit board 410 and mounted thereon with the sensor module 420, a process for electrically connecting the wireless power reception antenna 114 and the sensor module 10 (for example, a soldering process) may be omitted when compared to the above-described embodiment.

Accordingly, in the wireless power reception module 400 according to the present embodiment, a work process can be simplified to reduce a process cost.

In this case, the circuit board 410 may be a multilayer printed circuit board (PCB) in which a plurality of PCBs are stacked as illustrated in FIG. 12, and the plurality of PCBs may be formed of the same material or different materials.

That is, each of the plurality of PCBs may be a known flexible PCB (FPCB) or Frame Retardant 4 (FR4) PCB. As a non-restrictive example, the circuit board 410 may have a shape in which two FPCBs are disposed between two FR4 PCBs.

In this case, each of the two FPCBs may include an extension portion 412 extending to a predetermined length outward from a body of the circuit board 410, and the extension portion 412 may be a connection line for electrical connection with another component.

However, the total number of layers of the multilayer PCB is not limited thereto, and the circuit board may be a multilayer PCB having 2 to 8 layers, and the total number of layers may be appropriately changed according to design conditions.

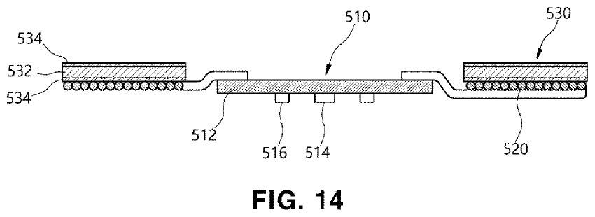

As another example, as illustrated in FIGS. 13 and 14, in the wireless power reception module 500 according to one embodiment of the present invention, the wireless power reception antenna 520 may be a flat coil, and both end portions of the flat coil may be connected to a circuit board 512 constituting the sensor module 510 through a soldering processing.

That is, the wireless power reception module 500 according to the present embodiment may include the circuit board 512 and the sensor module 510 including at least one sensor mounted on the circuit board 512, and the wireless power reception antenna 520 provided as a flat coil may be disposed outside the circuit board 512 to surround a circumference of the circuit board 512.

In this case, a shielding unit 530 for shielding a magnetic field may be disposed on one surface of the wireless power reception antenna 520.

Herein, the shielding unit 530 may include a magnetic sheet 532 having a plate shape for shielding the magnetic field, a protective film 534 attached to at least one surface of the magnetic sheet 532, and a through part formed to pass through the shielding unit 530 at a position corresponding to the sensor module 510.

In this case, the magnetic sheet 122 and the protective film 124 or 224a and 224b may be equally applied to the magnetic sheet 532 and the protective film 534.

Accordingly, in the wireless power reception module 500 according to the present embodiment, a total size of the circuit board 512 can be reduced as much as an amount of the circuit board 512 used for forming the wireless power reception antenna 520, that is, an area corresponding to the second region S2 in the circuit board 410 of the above-described embodiment, when compared to the wireless power reception module 400 of the above-described embodiment.

Accordingly, a production cost of the wireless power reception module 500 according to the present embodiment can be reduced.

That is, in the wireless power reception module 500 according to the present embodiment, the wireless power reception antenna 520 may be provided as the low-cost flat coil, and the flat coil may be electrically connected to the circuit board 512 constituting the sensor module 510 through the soldering process.

Accordingly, in the wireless power reception module 500 according to the present embodiment, the wireless power reception antenna 520 and the sensor module 510 can be integrated, and a use amount of relatively expensive circuit board can also be minimized to reduce the production cost.

As described above, in the wireless power reception module 400 or 500 according to one embodiment of the present invention, since a total use amount of the circuit board is minimized or a portion of the circuit board (the area of the first region S1 in FIG. 11) is fundamentally prevented from being discarded, a manufacturing cost can be reduced.

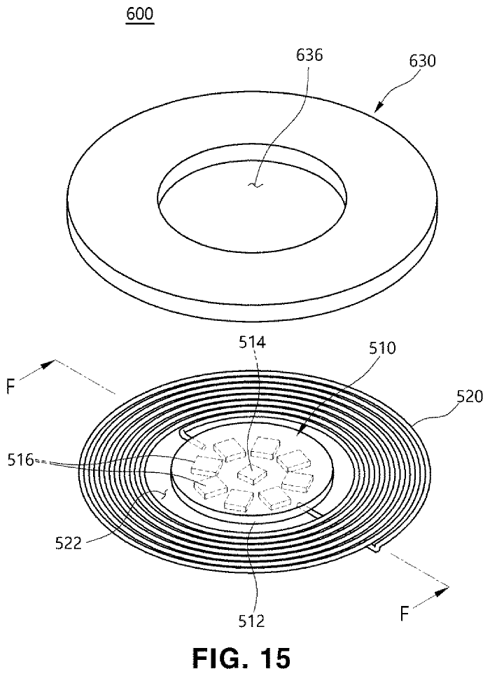

Meanwhile, the wireless power reception module 600 according to one embodiment of the present invention may include the wireless power reception antenna 520 and a shielding unit 630 disposed on one surface of the wireless power reception antenna 520 as illustrated in FIGS. 15 to 17.

In this case, the wireless power reception antenna 520 may be provided as a flat coil in which an empty space portion 522 formed in a central portion thereof, and the shielding unit 630 may include a through part 636 formed to pass through the shielding unit 630 at a position corresponding to the empty space portion 522.

In addition, the wireless power reception module 600 according to one embodiment of the present invention may further include the sensor module 510 disposed at a position corresponding to the empty space portion 522 and the through part 636.

Herein, the sensor module 510 may include the LED 514 and the photodiode 516 as described above, the sensor module 510 may be a heart rate sensor module, and the LED 514 and the photodiode 516 may be mounted on one surface of the circuit board 512 having a predetermined area.

Accordingly, the wireless power reception antenna 520 provided as the flat coil may be disposed outside the circuit board 512 to surround a circumference of the circuit board 512.

In this case, in a state in which the sensor module 510 is disposed at the position corresponding to the empty space portion 522 and the through part 636, both end portions of the flat coil may be connected to the circuit board 512 constituting the sensor module 510 through a soldering process.

Accordingly, the sensor module 510 may be integrated with the wireless power reception antenna 520.

Accordingly, in the wireless power reception module 600 according to the present embodiment, since the wireless power reception antenna 520 may be provided as the low-cost flat coil like in the wireless power reception module 500 of the above-described embodiment, the wireless power reception antenna 520 and the sensor module 510 can be integrally formed, a use amount of relatively expensive circuit board can be minimized, and a production cost can be reduced by minimizing the use amount of the circuit board.

In this case, when compared to the above-described embodiment, the wireless power reception module 600 according to one embodiment of the present invention may be formed so that a part of a magnetic field leaking toward the through part 636 may be induced toward the wireless power reception antenna 520.

To this end, the shielding unit 630 may include a magnetic member 632 formed of a magnetic material to shield a magnetic field, an accommodation groove 634 formed to be recessed in one surface of the magnetic member 632 to accommodate the wireless power reception antenna 520, and the through part 636 formed to pass through the magnetic member 632.

In addition, the magnetic member 632 may include a base 632a having a closed-loop shape in which the through part 636 is formed in a central portion of the base 632a, an inner protruding part 632b protruding to a predetermined thickness from one surface of the base 632a to surround the through part 636, and an outer protruding part 632c spaced a distance from the inner protruding part 632b and protruding to a predetermined thickness from one surface of the base 632a.

That is, the inner protruding part 632b may be formed in a closed-loop shape along an inner edge of the base 632a, and the outer protruding part 632c may be formed in a closed-loop shape along an outer edge of the base 632a.

Accordingly, the accommodation groove 634 may be formed between the inner protruding part 632b and the outer protruding part 632c which are disposed on one surface of the base 632a to be spaced apart from each other, and the accommodation groove 634 may be formed in a closed-loop shape of which one side is open like the base 632a, the inner protruding part 632b, and the outer protruding part 632c.

Herein, the base 632a, the inner protruding part 632b, and the outer protruding part 632c may be formed of the same magnetic material, and the base 632a, the inner protruding part 632b, and the outer protruding part 632c may be integrally formed.

As an example, a Mn—Zn ferrite sheet, a Ni—Zn ferrite sheet, a ribbon sheet formed of an amorphous or nano-crystalline alloy, a polymer sheet, a permalloy, or the like may be used as the magnetic member 632, and when the base 632a, the inner protruding part 632b, and the outer protruding part 632c are formed of the same magnetic material, the base 632a, the inner protruding part 632b, and the outer protruding part 632c may be integrally formed.

Accordingly, when the wireless power reception antenna 520 is inserted into the accommodation groove 634, the inner protruding part 632b may be positioned in the empty space portion 522, and the outer protruding part 632c may be disposed to surround an edge of the wireless power reception antenna 520.

Accordingly, in the wireless power reception module 600 according to one embodiment of the present invention, the base 632a may serve as a main shielding part which shields a main magnetic field induced to the wireless power reception antenna 520, and the inner protruding part 632b and the outer protruding part 632c may serve as auxiliary shielding parts which assist the base 632a.

That is, the inner protruding part 632b may reflect a part of a leakage magnetic field leaking through the through part 636 of the main magnetic field induced to the wireless power reception antenna 520 and induce the part of the leakage magnetic field toward the wireless power reception antenna 520, and the outer protruding part 632c may reflect a part of a leakage magnetic field, which leaks to the outside, of the main magnetic field induced to the wireless power reception antenna 520 and induce the part of the leakage magnetic field toward the wireless power reception antenna 520.

Accordingly, in the wireless power reception module 600 according to one embodiment of the present invention, even when the empty space portion 522 is formed in the central portion of the wireless power reception antenna 520, and the through part 636 is formed in a central portion of the shielding unit 630 in consideration of an arrangement relationship with the sensor module 510, a part of a magnetic field leaking to the outside through the through part 636 may be induced toward the wireless power reception antenna 520.

As a result, in the wireless power reception module 600 according to one embodiment of the present invention, the performance of the wireless power reception antenna 520 can be improved further.

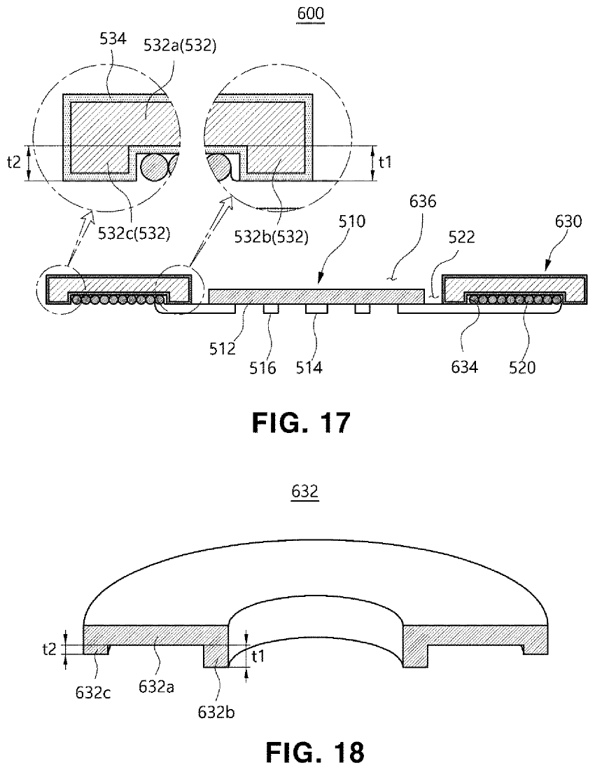

Herein, thicknesses t1 and t2 of the inner protruding part 632b and the outer protruding part 632c which protrude from one surface of the base 632a may be formed to be the same as illustrated in FIG. 17.

In addition, the inner protruding part 632b and the outer protruding part 632c which protrude from one surface of the base 632a may be formed to have different thicknesses. For example, as illustrated in FIG. 18, the inner protruding part 632b may be formed to protrude to a predetermined height from one surface of the base 632a so that the inner protruding part 632b has a thickness t1 which is greater than a thickness t2 of the outer protruding part 632c.

In addition, the thickness t1 of the inner protruding part 632b may be greater than or equal to a thickness of the wireless power reception antenna 520 inserted into the accommodation groove 634 so as to easily shield a magnetic field leaking to the through part 636.

In this case, in the wireless power reception module 600 according to the present embodiment, the shielding unit 630 may include an insulating protection layer 638 with a predetermined thickness formed on a surface of the magnetic member 632.

As an example, the insulating protection layer 638 may be a coating layer which covers an entire surface of the magnetic member 632 as illustrated in FIG. 17.

Accordingly, the wireless power reception antenna 520 inserted into the accommodation groove 634 can be prevented from being electrically short-circuited with the magnetic member 632 by the insulating protection layer 638, and in a state in which the wireless power reception antenna 520 is inserted into the accommodation groove 634, a possibility of a short circuit which may occur when electrically connected to another component can be prevented.

In addition, the insulating protection layer 638 can prevent the surface of the magnetic member 632 from being exposed to the outside. Accordingly, a possibility of breakage of the magnetic member 632 due to an external impact can be reduced, and powder or fine fragments can be prevented from separating from the surface of the magnetic member 632.

As a non-restrictive example, the insulating protection layer 638 may be formed of a polymer resin including at least one selected from the group of a wax, epoxy resin, melanin resin, silicone resin, acrylic resin, ethylene-propylene diene monomer (EPDM) resin, and polyvinyl alcohol (PVA) resin, and the insulating protection layer 638 may include an insulating filler.

However, a position at which the insulating protection layer 638 is formed is not limited thereto, and the insulating protection layer 638 may be provided to cover a partial surface of the entire surface of the magnetic member 632.

In addition, any known material used as an insulating coating layer may be used in addition to the above-described materials as the insulating protection layer 638.

Meanwhile, in the above description, although it has been described that the wireless power reception module 100, 200, 300, 400, 500, or 600 according to one embodiment of the present invention is the reception module for receiving the wireless power transmitted from the outside, the present invention is not limited thereto.

That is, in the wireless power reception module 100, 200, 300, 400, 500, or 600, when the wireless power reception antenna 114, 430, or 520 serves as a wireless power transmission antenna which transmits wireless power or is replaced with a wireless power transmission antenna which transmits wireless power, the wireless power reception module 100, 200, 300, 400, 500, or 600 may be implemented as a wireless power transmission module.

While some embodiments of the present invention have been described above, the spirit of the present invention is not limited to the embodiments proposed in this specification, and other embodiments may be easily suggested by adding, changing, and removing components within the scope of the invention by those skilled in the art and will fall within the spirit of the present invention.

1. A wireless power reception module comprising:
an antenna unit which includes a circuit board including a first surface and a second surface which are opposite to each other, a wireless power reception antenna formed as an antenna pattern on the first surface of the circuit board, a terminal pattern extending to a predetermined length from the wireless power reception antenna on the first surface of the circuit board, and a first coverlay attached to the first surface of the circuit board; and
a shielding unit which includes a magnetic sheet formed of a magnetic material to shield a magnetic field and having a side surface exposed to an outside and is disposed on one surface of the antenna unit to be positioned at a position corresponding to the wireless power reception antenna,
wherein the first coverlay is attached to the first surface to cover both the wireless power reception antenna and a part of a total length of the terminal pattern formed on the first surface.

2. The wireless power reception module of claim 1, wherein the magnetic sheet is a ribbon sheet formed of an amorphous alloy or a ribbon sheet formed of a nano-crystalline alloy.

3. The wireless power reception module of claim 1, wherein the first coverlay is formed of an insulating material.

4. The wireless power reception module of claim 1, wherein the shielding unit is disposed to be positioned on one surface of the first coverlay.

5. The wireless power reception module of claim 1, wherein the shielding unit includes:
a magnetic sheet having a plate shape and a predetermined area; and
a pair of protective films attached to both surfaces of the magnetic sheet so that a side surface of the magnetic sheet is exposed to the outside.

6. The wireless power reception module of claim 1, comprising an empty space portion having a predetermined area and formed to pass through the wireless power reception module to be positioned at a central portion of the wireless power reception antenna,
wherein the empty space portion includes a first empty space portion having a predetermined area and formed to pass through a central portion of the antenna unit and a second empty space portion having a predetermined area and formed to pass through a central portion of the shielding unit.

7. The wireless power reception module of claim 6, wherein the circuit board includes:
a body, in which the first empty space portion is formed in a central portion of the body and the wireless power reception antenna is formed as a pattern on at least one surface of the body; and
a protruding part protruding to a predetermined length from an inner edge of the body toward the first empty space portion,
wherein at least a portion including one end portion in the terminal pattern is formed on the circuit board so as to be positioned on the protruding part.

8. The wireless power reception module of claim 6, further comprising a sensor module disposed in the empty space portion,
wherein the sensor module includes a substrate member and a terminal pad formed on the substrate member, and
the wireless power reception module and the sensor module are integrated by the terminal pattern and the terminal pad electrically connected to each other through a solder member.

9. The wireless power reception module of claim 8, wherein the sensor module is a heart rate sensor module.

10. A wireless power reception module comprising:
a wireless power reception antenna provided with a flat coil in which an empty space portion having a predetermined area is formed in a central portion thereof; and
a shielding unit including a magnetic member formed of a magnetic material to shield a magnetic field, an accommodation groove formed to be recessed in one surface of the magnetic member to accommodate the wireless power reception antenna, a through part formed to pass through the magnetic member at a position corresponding to the empty space portion, and an insulating protection layer with a predetermined thickness formed on the surface of the magnetic member,
wherein the magnetic member includes a base which has a closed-loop shape and in which the through part is formed in a central portion of the base, an inner protruding part protruding to a predetermined thickness from one surface of the base to surround the through part, and an outer protruding part protruding to a predetermined thickness from the one surface of the base to be spaced apart from the inner protruding part so as to form the accommodation groove in the one surface of the base.

11. The wireless power reception module of claim 10, wherein the magnetic member is formed of a polymer material.

12. The wireless power reception module of claim 10, wherein the thicknesses of the inner protruding part and the outer protruding part that protrude from the one surface of the base are the same.

13. The wireless power reception module of claim 10, wherein the inner protruding part is formed to protrude from the one surface of the base to have a greater thickness than the outer protruding part.

14. The wireless power reception module of claim 10, wherein the base, the inner protruding part, and the outer protruding part are integrally formed.

15. The wireless power reception module of claim 10, further comprising a sensor module disposed in the empty space portion,
wherein both end portions of the flat coil are integrated with a circuit board constituting the sensor module through a soldering process.

16. The wireless power reception module of claim 15, wherein the sensor module is a heart rate sensor module.
